(19) United States
(12) Patent Application Publication
(10) Pub. No.: US 2024/0291228 A1
(43) Pub. Date: Aug. 29, 2024

(54) SYSTEM FOR EXPANDING IRRADIATION RANGE OF LASER LIGHT

(71) Applicant: OK Fiber Technology Co., Ltd., lbaraki (JP)

(72) Inventor: Kiyoshi OKA, Ibaraki (JP)

(73) Assignee: OK Fiber Technology Co., Ltd., Ibaraki (JP)

(21) Appl. No.: 18/571,633

(22) PCT Filed: Aug. 30, 2022
(86) PCT No.: PCT/JP2022/032627

§ 371 Date: Dec. 18, 2023

(30) Foreign Application Priority Data
Sep. 10, 2021 (JP) 2021-147863

(51) Int. Cl. H01S 5/02251; G02B 23/24

(52) U.S. Cl. CPC H01S 5/02251; G02B 23/2469; H01S 2301/18

(57) ABSTRACT

The system of the present invention comprises a first optical fiber for transmitting a laser light from a light source, a second optical fiber for irradiating a laser light, wherein the second optical fiber is a second optical fiber different from the first optical fiber, and an apparatus optically connected to the first optical fiber and the second optical fiber, wherein the apparatus comprises a housing having an inside space, a first lens disposed within the inside space, and a second lens disposed within the inside space, wherein the first lens and the second lens are arranged so that a laser light exited from the first optical fiber enters the second optical fiber through the second lens after going through the first lens, wherein the numerical aperture of the first lens and the first optical fiber is smaller than the numerical aperture of the second lens and the second optical fiber.

### TECHNICAL FIELD

The present invention relates to a system for expanding irradiation range of laser light.

### BACKGROUND ART

A fiberscope used for an endoscope or the like has been conventionally known (see, for example, Non Patent Literature 1).

### CITATION LIST

### Non Patent Literature

[NPL 1] OLYMPUS CORPORATION, “Faibasukopu no tanjou (Birth of fiberscope)”, [online], [retrieved on Aug. 24, 2021], Internet <URL: https://www.onaka-kenko.com/endoscope-closeup/endoscope-history/eh_03.html>

### SUMMARY OF INVENTION

### Technical Problem

The irradiation range of a laser light of a conventional fiberscope was not wide enough to be able to secure a field of operation. While it is possible to expand the irradiation scope of a laser light by providing a lens to a light irradiation part of a fiberscope in order to extend the irradiation scope of a laser light, a large lens is required in order to do so. As a result, there had been the problem of the fiberscope itself being large, which makes it difficult to carry out examination using the fiberscope.

In addition, a conventional fiberscope had unevenness in the irradiation intensity of a laser light.

The present invention was invented in view of the above-mentioned problem, and the purpose is to provide a system for expanding the irradiation range of a laser light and achieving even irradiation intensity of a laser light while avoiding increase in size of the apparatus.

### Solution to Problem

In one aspect of the present invention, the system of the present invention is a system for expanding an irradiation range of a laser light, the system comprising: a first optical fiber for transmitting a laser light from a light source; a second optical fiber for irradiating the laser light, wherein the second optical fiber is different from the first optical fiber; and an apparatus optically connected to the first optical fiber and the second optical fiber, the apparatus comprising: a housing having an inside space; a first lens disposed within the inside space; and a second lens disposed within the inside space, wherein the first lens and the second lens are disposed so that the laser light exited from the first optical fiber would enter the second optical fiber through the second lens after going through the first lens, and wherein the numerical aperture of the first lens and the first optical fiber is smaller than the numerical aperture of the second lens and the second optical fiber.

In one embodiment of the present invention, the numerical aperture of the first lens may be substantially the same as the numerical aperture of the first optical fiber, and the numerical aperture of the second lens may be substantially the same as the numerical aperture of the second optical fiber.

In one embodiment of the present invention, the numerical aperture of the first lens and the first optical fiber may be smaller by about 0.5 or greater than the numerical aperture of the second lens and the second optical fiber.

In one embodiment of the present invention, the numerical aperture of the first lens and the first optical fiber may be about 0.2, and the numerical aperture of the second lens and the second optical fiber may be about 0.7 or greater.

In one embodiment of the present invention, the first lens may be an achromatic lens.

In one embodiment of the present invention, the second lens may be an aspherical lens configured to focus the laser light that entered the second lens to the second connector.

In one embodiment of the present invention, the apparatus further comprises a filter for restricting a laser light that can pass through, wherein the filter may be disposed between the first lens and the second lens.

In one embodiment of the present invention, the filter may be at least any one of a band-pass filter, a low-pass filter, or a high-pass filter.

In one embodiment of the present invention, the filter may be disposed tilted with respect to an optical axis of the laser light.

In one embodiment of the present invention, the apparatus further comprises a light quantity distribution correction optical system consisting of a pair of combined lenses for achieving even light quantity distribution of a laser light than can pass through, wherein the light quantity distribution correction optical system may be disposed between the first lens and the second lens.

In one embodiment of the present invention, an optical axis of the second lens may dispose the second lens so as to be at a position deviated from an optical axis of the first lens.

In one embodiment of the present invention, the first optical fiber may be a mode mixing fiber.

In one embodiment of the present invention, a light irradiation part of the second optical fiber may not be installed with a lens for expanding an irradiation range of the laser light.

In one embodiment of the present invention, an irradiation range of the laser light that passed through the apparatus may be at least five-folds in size over an irradiation range of a laser light that had not passed through the apparatus.

In one embodiment of the present invention, a maximum value of an irradiation intensity of the laser light that passed through the apparatus may be about 60% or less of a maximum value of an irradiation intensity of a laser light that had not passed through the apparatus.

In one embodiment of the present invention, the system further comprises the light source, wherein the light source may be a white color laser light source for illumination.

In one embodiment of the present invention, an optical axis within the apparatus and an optical axis of the second optical fiber may intersect.

In one embodiment of the present invention, the second optical fiber may be an optical fiber installed in an endoscope.

### Advantageous Effects of Invention

According to the present invention, it is possible to provide a system for expanding an irradiation range of a laser light and achieving even irradiation intensity of a laser light while avoiding increase in size of the apparatus.

### BRIEF DESCRIPTION OF DRAWINGS

FIG. 1A is a diagram showing an example of a configuration of a new system 100 that contributes to the irradiation range and the irradiation intensity of a laser light.

FIG. 1B is a diagram showing another example of a configuration of the apparatus 110.

FIG. 1C is a diagram showing another example of a configuration of the apparatus 110.

FIG. 1D is a diagram showing another example of a configuration of the apparatus 110.

FIG. 1E is a diagram showing another example of a configuration the apparatus 110.

FIG. 1F is a diagram showing another example of a configuration of the apparatus 110.

FIG. 2A is a diagram showing an implementation example of the apparatus 110.

FIG. 2B is a diagram showing another implementation example of the apparatus 110.

FIG. 3A is a diagram showing the result of a comparison experimentation in accordance with the presence/absence of the use of the apparatus 110.

FIG. 3B is a diagram showing the result of another comparison experimentation in accordance with the presence/absence of the use of the apparatus 110.

FIG. 4A is a diagram showing another implementation example of the apparatus 110.

FIG. 4B is a diagram showing another implementation example of the apparatus 110.

### DESCRIPTION OF EMBODIMENTS

The terms used herein are defined below.

Unless explicitly shown in a different form, the term “about” refers to +10% of the value shown right after.

The term “substantially the same” refers to a difference being 0.1 or less when used regarding the numerical aperture.

The embodiment of the present invention is explained below while referring to the drawings. Furthermore, the same reference number is used for the same component throughout the drawings.

### 1. A New Optical System That Contributes to the Irradiation Range and the Irradiation Intensity of a Laser Light

The Applicant proposes a new optical system that contributes to the irradiation range and the irradiation intensity of a laser light. The intention of this new optical system is to enable expansion of the irradiation range of a laser light and achievement of substantial evenness of the irradiation intensity of a laser light while avoiding increase in size of the apparatus. According to this new optical system, it is possible for a laser light transmitted through a first optical fiber having a first numerical aperture to go through a first lens having the first numerical aperture, further go through a second lens having a second numerical aperture greater than the first numerical aperture, and focus to a second optical fiber having the second numerical aperture, so as to irradiate a laser light having a wider irradiation range and a more even irradiation intensity to an irradiated location.

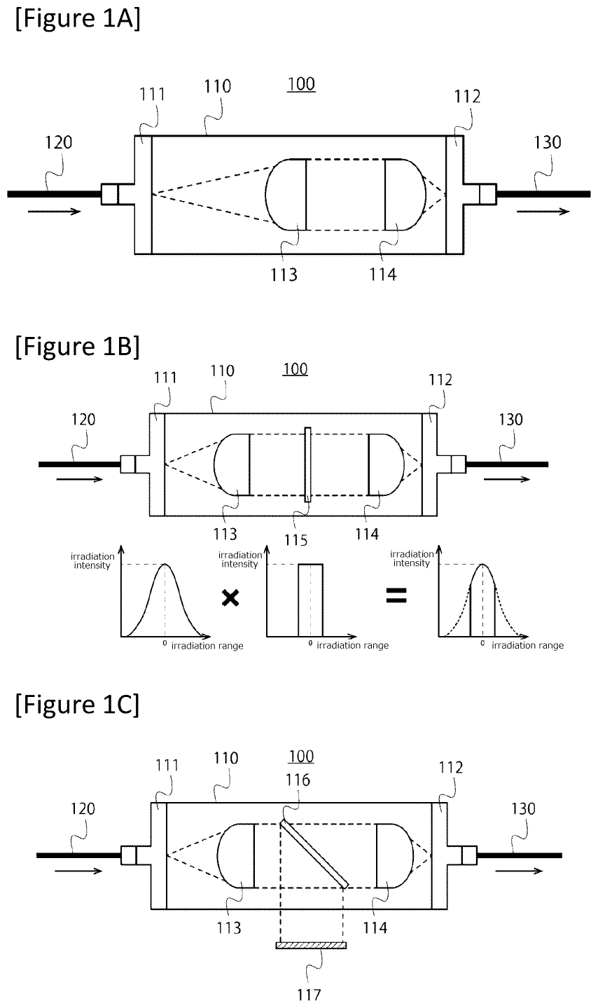

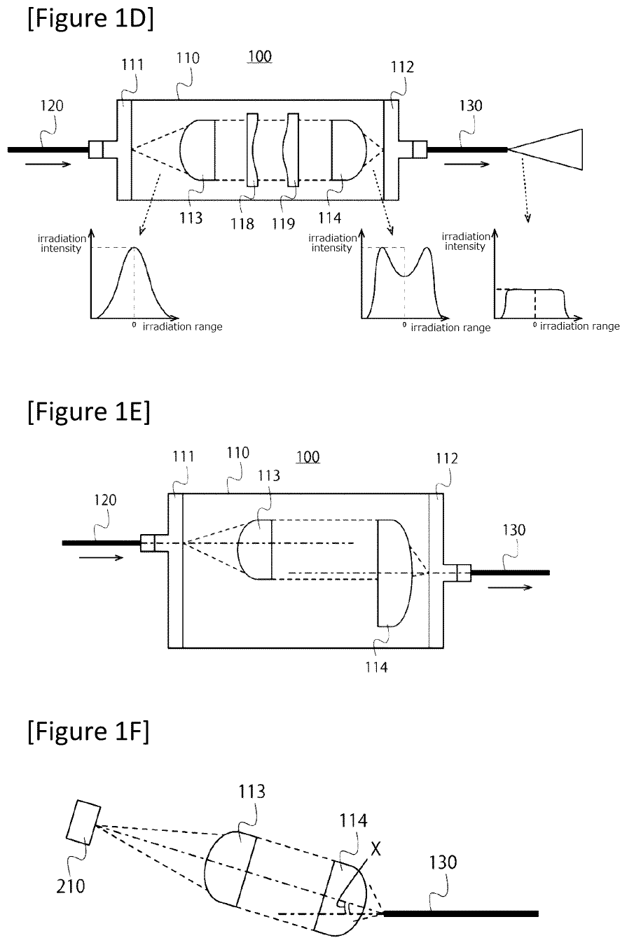

FIG. 1A shows an example of a configuration of a new system 100 that contributes to the irradiation range and the irradiation intensity of a laser light.

In the embodiment shown in FIG. 1A, the system 100 comprises: an apparatus 110 that contributes to the irradiation range and the irradiation intensity of a laser light; an optical fiber 120 (first optical fiber) for transmitting a laser light from a light source; and an optical fiber 130 (second optical fiber) that comprises a light irradiation part for irradiating a laser light. The light source may be any light source. In one embodiment, the light source is a laser light source for illumination. In addition, in FIG. 1A, there is one optical fiber 120 and one optical fiber 130, but the present invention is not limited thereto. The number of each of the optical fiber 120 and the optical fiber 130 may be any number. For example, at least one of the optical fiber 120 and the optical fiber 130 may be plural.

The apparatus 110 comprises a housing that comprises a connector 111 optically connected to the optical fiber 120, and a connector 112 optically connected to the optical fiber 130. The connector: 111 and the connector 112 may be configured to be exchangeable. The connector 111 and the connector 112 are, for example, but not limited to, an FC connector or an SMA connector.

The apparatus 110 further comprises a lens 113 and a lens 114 within the housing of the apparatus 110. The lens 113 is disposed more proximal to the connector 111 than to the connector 112 within the housing of the apparatus 110, and the lens 114 is disposed more proximal to the connector 112 than to the connector 111 within the housing of the apparatus 110. In other words, the lens 113 and the lens 114 are disposed so that a laser light received in the connector 111 through the optical fiber 120 would enter the connector 112 through the lens 114 after going through the lens 113. The lens 113 may be, for example, an achromatic lens. The lens 114 may be, for example, an aspherical lens configured to focus a laser light received in the lens 114 to the connector 112.

The numerical aperture of the lens 113 may be substantially the same as the numerical aperture of the optical fiber 120, and the numerical aperture of the lens 114 may be substantially the same as the numerical aperture of the optical fiber 130. The numerical aperture of the optical fiber 120 and the numerical aperture of the lens 113 are smaller than the numerical aperture of the lens 114 and the numerical aperture of the optical fiber 130. The numerical aperture of the optical fiber 120 and the numerical aperture of the lens 113 may be, for example, about 0.2, more preferably may be about 0.22. the numerical aperture of the lens 114 and the numerical aperture of the optical fiber 130 may be, for example, about 0.5 or greater, preferably may be about 0.7 or greater, most preferably may be about 0.87. Therefore, the maximum irradiation angle θ of a laser light that is transmitted to an optical fiber via the apparatus 110 and irradiated in the atmosphere may be about 30° or greater, preferably may be about 44.4°, most preferably may be about 60.5°.

The maximum irradiation angle θ of a laser light irradiated from an optical fiber: in an atmosphere is generally sought using inverse trigonometric function with the numerical aperture NA of the optical fiber as a variable.

Since a laser light as conventionally irradiated from an optical fiber with a numerical aperture of about 0.22, the maximum irradiation angle θ of the laser light was sin⁻¹ (0.22)=about 12.7°. Meanwhile, the present invention extends the irradiation range of a laser light using the apparatus 110 of the present invention to enable irradiation of the laser light using, for example, an optical fiber with a numerical aperture of about 0.87, and thus the maximum irradiation angle θ of the laser light would be sin⁻¹ (0.87)=about 60.5°. The irradiation range of a laser light that passed through the apparatus 110 would have a size that is about 61-folds of an irradiation range of a laser light of the case of not passing the apparatus 110 (i.e., a laser light that had not went through the apparatus 110 and had not undergone any processing). However, the present invention is not limited thereto. Various irradiation ranges of expansion can be selected by adjusting the size of the numerical aperture of the lens 114 and the numerical aperture of the optical fiber 130 to the numerical aperture of the lens 113 and the optical fiber 120. For example, the irradiation range of a laser light that passed through the apparatus 110 may be a size of at least about 5-folds, at least about 6-folds, at least about 7-folds, at least about 8-folds, at least about 9-folds, at least about 10-folds, at least about 15-folds, at least about 20-folds, at least about 30-folds, at least about 40-folds, at least about 50-folds, at least about 60-folds of the irradiation range of a laser light of the case of not passing the apparatus 110 (i.e., a laser light that had not went through the apparatus 110 and had not undergone any processing).

Accordingly, it is possible to expand the irradiation range of a laser light irradiated from the light irradiation part of the optical fiber 130 by temporarily extending the laser light using the lens 113 having a smaller numerical aperture and focusing the laser light that had passed through the lens 113 to the optical fiber 130 using the lens 114 having a larger numerical aperture.

In addition, it is possible to irradiate a laser light having a top-hat-type irradiation intensity (i.e., a smoothened irradiation intensity with substantially the same irradiation intensity of a laser light within a certain irradiation range) as schematically shown in the graph below with the configuration of the apparatus 110 shown in FIG. 1A. This enables irradiation of a laser light to a irradiated location in a wide range and without any unevenness.

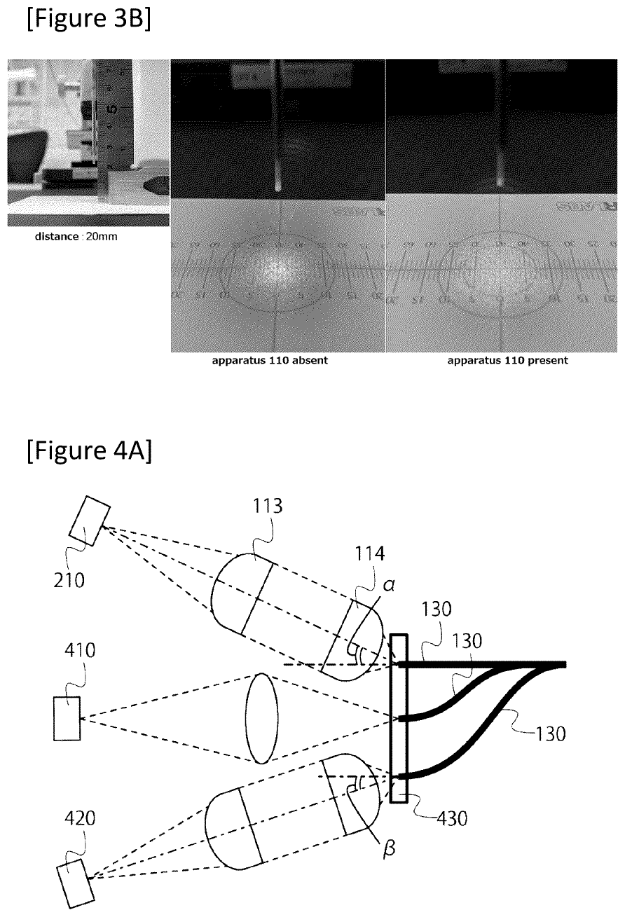

The above-described graphs use the horizontal axis for the irradiation range and use the vertical axis for the irradiation intensity. In addition, the above-described graphs use the irradiation location, which is at the shortest distance from the light irradiation part of the optical fiber, as a criterion (zero) of the irradiation range. When a laser light was irradiated from a conventional optical fiber, irradiation having a Gaussian distribution-type profile was observed. Since irradiation having a Gaussian distribution-type profile causes unevenness in the irradiation of the laser light (i.e., the irradiation intensity is too high near the criterion point of the irradiation range and it is very bright, and the irradiation intensity is too small at a position away from the criterion point of the irradiation range and it is dim), the irradiation is not preferable especially for use in a fiberscope. In this regard, the present invention would irradiate a laser light from an optical fiber via the apparatus 110, which can realize irradiation having a top-hat-type profile (see FIG. 3A and FIG. 3B). Since it is possible to irradiate a laser light towards an irradiated location (e.g., a treatment site within a body of a human) at a constant irradiation intensity throughout a constant irradiation range, it is possible to, for example, relatively evenly illuminate the irradiated location when using the system for illumination purposes. The maximum value of the irradiation intensity of a laser light that underwent processing by the apparatus 110 may be about 40% or lower, about 45% or lower, about 50% or lower, about 55% or lower, about 60% or lower, about 65% or lower, about 70% or lower, about 75% or lower, about 80% or lower, or about 90% or lower of the maximum value of the irradiation intensity of an unprocessed laser light (i.e., a laser light that does not pass the apparatus 100 and had not undergone any processing).

In the embodiment shown in FIG. 1A, the light irradiation part provided to the tip part of the optical fiber 130 (e.g., the tip part of an endoscope) does not comprise an optical system (e.g., a lens) that can expand the irradiation range of a laser light and an optical system (e. g., lens) that can at least partially adjust the irradiation intensity of a laser light.

In addition, in the embodiment shown in FIG. 1A, the apparatus 110 may further comprise a filter (not shown) for restricting a laser light that can pass through between the lens 113 and the lens 114. For example, the filter between the lens 113 and the lens 114 may be a filter that restricts the extension of the wavelength of a laser light source. The filter is, for example, but not limited to, a band-pass filter, a low-pass filter, a long-pass filter, or the like. When the filter is a band-pass filter, it is possible to selectively permeate only a laser light having a desired wavelength. For example, even if a laser element with a great width of wavelength is used, it is possible to narrow the wavelength. In addition, since a laser light is temporarily extended by the lens 113, the filter would not be damaged even when a high output laser light is used in the apparatus 110.

FIG. 1B shows another example of the configuration of the apparatus 110. In the embodiment shown in FIG. 1B, the apparatus 110 further comprises a filter 115 that only passes through a laser light having a predetermined irradiation intensity between the lens 113 and the lens 114 to restrict the extension of the wavelength of a laser light source. Such a filter 115 may be, for example, disposed perpendicularly to the optical axis of the lens 113 and the lens 114. This can suppress the extension of the wavelength of the laser light and cause a laser light of less than a predetermined irradiation intensity to not reach the lens 114, which thus enables reduction of noise of the laser light caused in a fluorescence observation image.

FIG. 1C shows another example of the configuration of the apparatus 110. In the embodiment shown in FIG. 1C, the apparatus 110 further comprises a filter 116 that permeates at least a part of a laser light that passed through the lens 113 and reflects the remaining between the lens 113 and the lens 114 to restrict the extension of the wavelength of a laser light source. Such a filter 116 may be disposed so as to be angled with respect to the optical axis of the lens 113 and the lens 114. This can avoid oscillation of a laser light from being unstable by a laser light reflected from the filter 116 returning to the lens 113 side without permeating the filter 116. In other words, it is possible to stabilize the oscillation of a laser light and suppress generation of a stray light inside the optical system. A laser light that reflected without permeating the angled filter 116 may, for example, proceed to a direction that is perpendicular to the optical axis of the lens 113 and the lens 114 to be absorbed by an absorber 117. In the embodiment shown in FIG. 1C, the apparatus 110 may also comprise an absorber 117.

FIG. 1D shows another example of the configuration of the apparatus 110. In the embodiment shown in FIG. 1D, the apparatus 110 further comprises a light quantity distribution correction optical system (a pair of combined lenses 118 and 119) that makes correction so that the irradiation intensity of a laser light that had been turned into a parallel light would be even. The pair of combined lenses 118 and 119 can mold the irradiation intensity distribution of a laser light to be a concave as shown in FIG. 1D. This enables correction of the light quantity of the periphery that is reduced in an output optical fiber (i.e., regarding the arrangement direction of a laser exit end, increase the density of a light ray in a portion apart from the optical axis while reducing the density of a light ray in a portion close to the optical axis of the lens, and regarding a direction that is perpendicular to the arrangement direction of the laser exit end, pass through a laser light as it is) so that the irradiation intensity distribution at the irradiated location would be close to even (flat). As such, since the irradiated light quantity distribution would be even, the light emission intensity of a fluorescence would be constant, enabling the boundary of the irradiated location to be readily understood. In addition, since it is possible to reduce the irradiation intensity around the criterion (zero) of the irradiation range, it is possible to suppress temperature rise of the irradiated location and/or deterioration of an agent at the irradiated location.

FIG. 1E shows another example of the configuration of the apparatus 110. In the embodiment shown in FIG. 1E, the optical axis of the lens 113 is relatively offset with respect to the optical axis of the lens 114. This enables a laser light that went through the lens 114 to enter the optical fiber 130 from a direction angled with respect to the optical axis of the optical fiber 130. It is possible to suppress the periphery light quantity reduction of the optical fiber 130 due to the increase in the component of light with a large incident angle.

In the embodiment shown in FIG. 1E, an example in which the optical axis of the lens 113 and the optical axis of the lens 114 are deviated while being parallel from one another has been explained, but the present invention is not limited thereto. For example, as shown in FIG. 1F, the optical axis of the lens 113 and the optical axis of the lens 114 may be angled so as to form an angle X with respect to the optical axis of the optical fiber 130 (i.e., the optical axis of the lens 113 and the optical axis of the lens 114 may be positioned so as to intersect with the optical axis of the optical fiber 130).

In addition, the optical fiber 120 in FIG. 1A may be, for example, a mode mixing fiber. This enables the irradiation intensity distribution of a laser light entering the inside of the apparatus 110 to be flattened. In this case, the periphery light quantity reduction inside the apparatus 110 and in the optical fiber 130 cannot be corrected, but a certain degree of improvement can be expected.

### 2. Implementation Example of the Apparatus 110

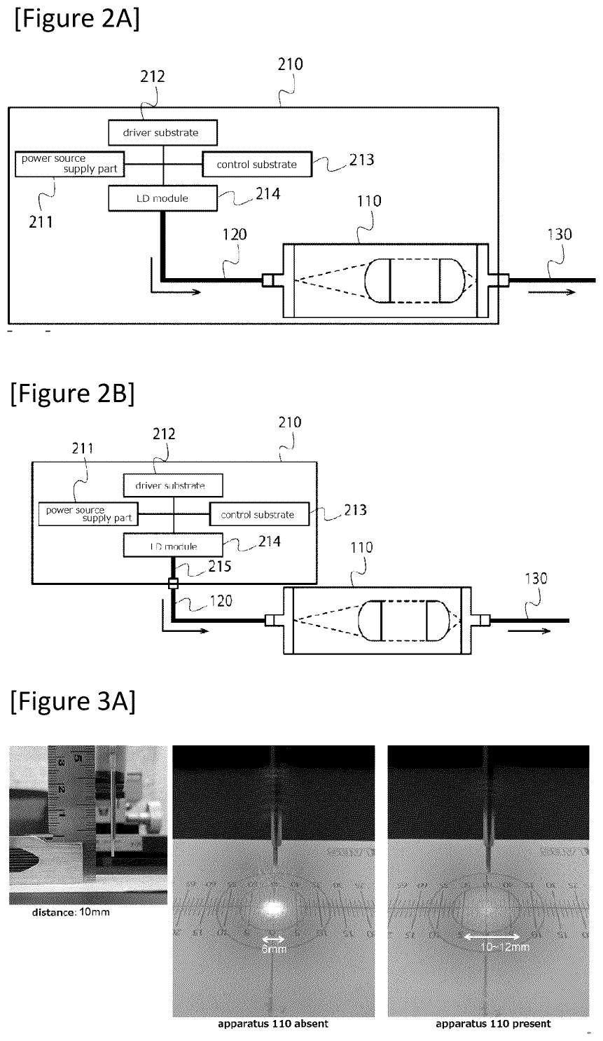

FIG. 2A shows an implementation example of the apparatus 110.

In the embodiment shown in FIG. 2A, the apparatus 110 is installed inside a laser oscillation apparatus 210 for oscillation of a laser light. In the embodiment shown in FIG. 2A, the laser oscillation apparatus 210 comprises a power source supply part 211 for supplying power from a power source (not shown), a driver substrate 212 that is driven by the power from the power source supply part 211, a control substrate 213 that controls the operation of the driver substrate 212, and an LED module 214 (light source) that oscillates a laser light with the driver substrate 212.

A laser light oscillated from the LD module 214 enters the apparatus 110 through the optical fiber 120. Within the apparatus 110, a laser light that entered the apparatus 110 is extended by permeating the lens 113, enters the lens 114, and focuses to the optical fiber 130 having a greater diameter and numerical aperture compared to the optical fiber 120. The laser light focused to the optical fiber 130 is transmitted through the optical fiber 130 and irradiated from the light irradiation part of the optical fiber 130 towards the irradiated location.

As such, it is possible to realize a laser oscillation apparatus configured to enable oscillation of a laser light with a wide irradiation range and substantially even irradiation intensity by providing the apparatus 110 inside the laser oscillation apparatus 210.

FIG. 2B shows another implementation example of the apparatus 110.

In the embodiment shown in FIG. 2B, the apparatus 110 is installed on the outside of the laser oscillation apparatus 210. In the embodiment shown in FIG. 2B, the laser oscillation apparatus 210 further comprises an optical fiber 215 that connects an LD module 214 and an optical fiber 120. Furthermore, the optical fiber 120 and the optical fiber 215 may be connected via a connector (e.g., an FC connector, an SMA connector).

As such, it is possible to realize irradiation of a laser light with a wide irradiation range and substantially even irradiation intensity without depending on the performance of the laser oscillation apparatus 210 by providing the apparatus 110 outside the laser oscillation apparatus 210.

Furthermore, in the embodiment shown in FIG. 2B, an example of connecting the optical fiber 215 extending from the LD module 214 and the optical fiber 120 extending from the apparatus 110 has been explained, but the present invention is not limited thereto. For example, the optical fiber 120 extending from the apparatus 110 may be an optical fiber equipped to the LD module 214 (i.e., the apparatus 110 and the LD module 214 may be connected to one another by the optical fiber 120 alone).

The apparatus 110 shown in FIG. 1A to FIG. 2B can be implemented in an endoscope system. In this case, the second optical fiber may be an optical fiber installed in an endoscope. It is possible to secure a field of operation during endoscopic surgery in a wider and more vivid manner by employing the apparatus 110 in the endoscope system. In addition, when taking a test drug in which a lesion portion reacts to light within a body, it is possible to carry out lesion observation in a wide range in a more efficient manner.

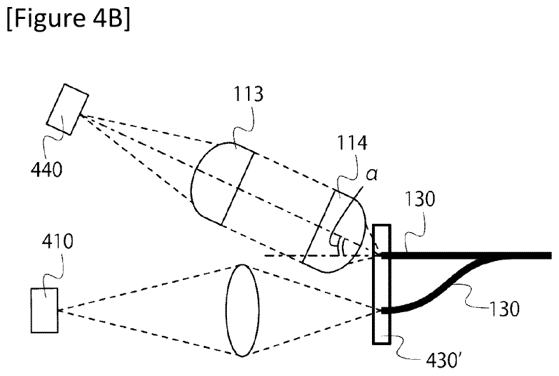

FIG. 4A shows another implementation example of the apparatus 110. In the embodiment shown in FIG. 4A, an optical system of a laser light of the apparatus 110, an optical system of an image of an image sensor 410, and an optical system of an illumination light of an illumination light source 420 (e.g., LED) are connected to the optical fiber 130 via a triple connector 430. In the embodiment shown in FIG. 4A, the optical system of the laser light of the apparatus 110 is positioned to form an angle α with respect to the optical axis of the optical fiber 130 corresponding to the optical system of the laser light of the apparatus 110. In addition, the optical system of the illumination light of the illumination light source 420 is positioned to form an angle β with respect to the optical axis of the optical fiber 130 corresponding to the optical system of the illumination light of the illumination light source 420. By forming such angles α and β, it is possible to attempt to save more space and it is possible to attempt to miniaturize a connector compared to the case of disposing the optical system of the laser light of the apparatus 110, the optical system of an image of the image sensor 410, and the optical system of the illumination light of the illumination light source 420 (e.g., LED) to be parallel to one another. In addition, by forming angles α and β, the components of a light with a great incidence angle increase, and it is possible to suppress periphery light quantity reduction of the optical fiber 130.

FIG. 4B shows another implementation example of the apparatus 110. In the embodiment shown in FIG. 4B, an optical system combining the optical system of the laser light of the apparatus 110 and the optical system of the illumination light of the illumination light source 420 (e.g., LED) (i.e., an optical system of a laser light+illumination light of an apparatus 440 that can oscillate a laser light source+illumination light source) and an optical system of an image of the image sensor 410 are connected to the optical fiber 130 via a double connector 430′. By utilizing an optical system combined in such a manner, it is possible to attempt to save more space, and it is possible to attempt further miniaturization of a connector.

### EXAMPLES

FIG. 3A shows 41 a result of a comparison experimentation in accordance with the presence/absence of the use of the apparatus 110.

The embodiment shown in FIG. 3A shows a result of an experimentation of irradiating a laser light from a height that is about 10 mm apart from the irradiated location. Furthermore, the numerical aperture of the first optical fiber is about 0.22, the core diameter of the first optical fiber is about 105 μm, the numerical aperture of the second optical fiber is about 0.87, and the core diameter of the second optical fiber is about 120 μm. When a laser light is irradiated with a conventional method that does not use the apparatus 110, the laser light was focused to the center of the irradiation position, and the diameter of the irradiation range of the laser light was about 6 mm. On the other hand, when a laser light is irradiated with the method of the present invention using the apparatus 110, the laser light was evenly irradiated around the center of the irradiation position, and the diameter of the irradiation range of the laser light was expanded to about 12 mm.

FIG. 3B shows a result of another comparison experimentation in accordance with the presence/absence of the use of the apparatus 110.

The embodiment shown in FIG. 3B shows a result of an experimentation of irradiating a laser light from a height that is about 20 mm apart from the irradiated location. The numerical aperture of the first optical fiber is about 0.22, the core diameter of the first optical fiber is about 105 μm, the numerical aperture of the second optical fiber is about 0.87, and the core diameter of the second optical fiber is about 120 μm. In the same manner as the embodiment shown in FIG. 3A, the case of irradiating a laser light with the method of the present invention using the apparatus 110 achieved more even irradiation of the laser light around the center of the irradiation position and had wider irradiation range of the laser light compared to the case of irradiating a laser light with a conventional method that does not use the apparatus 110.

Accordingly, the present invention has been exemplified using a preferable embodiment of the present invention, but the interpretation of the present invention should not be limited to this embodiment. It is understood that the scope of the present invention should be interpreted by the Claims alone. It is understood that those skilled in the art can practice an equivalent scope based on the description of the present invention and common general knowledge from the description of the specific and preferable embodiment of the present application.

### INDUSTRIAL APPLICABILITY

The present invention is useful as an invention providing a system or the like for expanding the irradiation range of a laser light and achieving even irradiation intensity of a laser light while avoiding increase in size of the apparatus.

### REFERENCE SIGNS LIST

100 system
110 apparatus
120 optical fiber
130 optical fiber
111 connector
112 connector
113 lens
114 lens

1. A system for expanding an irradiation range of a laser light, the system comprising:
a first optical fiber for transmitting a laser light from a light source;
a second optical fiber for irradiating the laser light, wherein the second optical fiber is different from the first optical fiber; and
an apparatus optically connected to the first optical fiber and the second optical fiber, the apparatus comprising:
a housing having an inside space;
a first lens disposed within the inside space; and
a second lens disposed within the inside space,
wherein the first lens and the second lens are disposed so that the laser light exited from the first optical fiber would enter the second optical fiber through the second lens after going through the first lens, and
wherein the numerical aperture of the first lens and the first optical fiber is smaller than the numerical aperture of the second lens and the second optical fiber.

2. The system of claim 1, wherein the numerical aperture of the first lens is substantially the same as the numerical aperture of the first optical fiber, and
wherein the numerical aperture of the second lens is substantially the same as the numerical aperture of the second optical fiber.

3. The system of claim 1, wherein the numerical aperture of the first lens and the first optical fiber is smaller by about 0.5 or greater than the numerical aperture of the second lens and the second optical fiber.

4. The system of claim 3, wherein the numerical aperture of the first lens and the first optical fiber is about 0.2, and the numerical aperture of the second lens and the second optical fiber is about 0.7 or greater.

5. The system of claim 1, wherein the first lens is an achromatic lens.

6. The system of claim 1, having a first connector optically connected to the first optical fiber and a second connector optically connected to the second optical fiber.
wherein the second lens is an aspherical lens configured to focus the laser light that entered the second lens to the second connector.

7. The system of claim 1, wherein the apparatus further comprises a filter for restricting a laser light that can pass through, wherein the filter is disposed between the first lens and the second lens.

8. The system of claim 7, wherein the filter is at least any one of a band-pass filter, a low-pass filter, or a high-pass filter.

9. The system of claim 7, wherein the filter is disposed tilted with respect to an optical axis of the laser light.

10. The system claim 1, wherein the apparatus further comprises a light quantity distribution correction optical system consisting of a pair of combined lenses for achieving even light quantity distribution of a laser light than can pass through, wherein the light quantity distribution correction optical system is disposed between the first lens and the second lens.

11. The system of claim 1, wherein an optical axis of the second lens disposes the second lens so as to be at a position deviated from an optical axis of the first lens.

12. The system of claim 1, wherein the first optical fiber is a mode mixing fiber.

13. The system of claim 1, wherein a light irradiation part of the second optical fiber is not installed with a lens for expanding an irradiation range of the laser light.

14. The system of claim 1, wherein an irradiation range of the laser light that passed through the apparatus is at least five-folds in size over an irradiation range of a laser light that had not passed through the apparatus.

15. The system of claim 1, wherein a maximum value of an irradiation intensity of the laser light that passed through the apparatus is about 60% or less of a maximum value of an irradiation intensity of a laser light that had not passed through the apparatus.

16. The system of claim 1, wherein the system further comprises the light source,
wherein the light source is a white color laser light source for illumination.

17. The system of claim 1, wherein an optical axis within the apparatus and an optical axis of the second optical fiber intersect.

18. The system of claim 1, wherein the second optical fiber is an optical fiber installed in an endoscope.

19. The system of claim 1, wherein a core diameter of the first optical fiber is about 105 μm, and a core diameter of the second optical fiber is about 120 μm.

20. The system of claim 1, wherein the apparatus further comprises a light quantity distribution correction optical system consisting of a pair of combined lenses for achieving even light quantity distribution of a laser light than can pass through, wherein the light quantity distribution correction optical system is disposed between the first lens and the second lens.
wherein an irradiation intensity distribution of the laser light that passed through the light quantity distribution correction optical system would be a concave.
